(19) United States
(12) Patent Application Publication
(10) Pub. No.: US 2014/0276197 A1
(43) Pub. Date: Sep. 18, 2014

(54) APPARATUS AND METHOD FOR DETERMINING TREATMENT ENDPOINTS FOR ALLERGEN TESTING

(71) Applicant: GREER LABORATORIES, INC., LEOIR, NC (US)

(72) Inventors: TOM GRIER, STATESVILLE, NC (US); DONNA REKKERTH, SCOTTSVILLE, NY (US); MARTY JOHNSON, GRANITE FALLS, NC (US); TIFFANY AHLERS, HICKORY, NC (US); CURTIS ANDREWS, BLOWING ROCK, NC (US)

(73) Assignee: GREER LABORATORIES, INC., LEOIR, NC (US)

(21) Appl. No.: 14/212,440

(22) Filed: Mar. 14, 2014

Related U.S. Application Data
(60) Provisional application No. 61/790,314, filed on Mar. 15, 2013.

(51) Int. Cl. A61B 17/20; A61B 5/00

(52) U.S. Cl. CPC A61B 17/205; A61B 5/7264; A61B 5/7282
USPC 600/556

(57) ABSTRACT

A system, a method, and an apparatus are described herein for determining skin test sensitivities. A first skin test to evaluate a patient's skin reaction to one or more allergens may he performed. After measurements are taken of any reactions to the first skin test, a second skin test may be performed based on the results of the first skin test. Based on the results of the second test, an appropriate treatment endpoint for the patient may be determined. A computer-implemented method may be used for determining skin test sensitivities that may guide a user through one or more appropriate test selection and treatment endpoint determinations.

### CROSS REFERENCE TO PRIOR APPLICATIONS

This application claims priority under 35 U.S.C. §119(e) to U.S. Provisional Patent Application No. 61/790,314, filed Mar. 15, 2013, titled “APPARATUS AND METHOD FOR DETERMINING TREATMENT ENDPOINTS FOR ALLERGEN TESTING,” the disclosure of which is hereby expressly incorporated herein by reference in its entirety.

### BACKGROUND

The present disclosure relates generally to an apparatus, a system, and a method for allergen testing.

A variety of types of allergy skin testing techniques have been used in the past. For example, intradermal dilution testing (IDT) has been used in which a series of dilutions are used progressively to quantify the degree of sensitivity to specific antigens. However, multiple intradermal skin tests can be uncomfortable to patients.

Modified quantitative testing (MQT) has also been used wherein an initial skin prick test is followed by a single intradermal test for certain allergens to further identify the level of sensitivity and to quantify an allergic response. However, current MQT tests fail to take into consideration fractional measurements of skin test wheals. Moreover, traditionally used skin tests rely on a practitioner administering the tests to properly categorize the test results in order to select a treatment endpoint. It would be desirable to have an automated method of determining or facilitating determination of treatment endpoints that take into consideration fractional wheals.

### SUMMARY OF THE INVENTION

According to some aspects of the disclosure, a computer-implemented method for determining a treatment endpoint for one or more allergens is described herein. The method includes receiving an input representing results of a first allergy test; determining, based on the results of the first allergy test, a second allergy test to be administered; receiving an input representing results of the second allergy test; and determining, via a processor, a treatment endpoint for the one or more allergens based on the results of the first allergen test and the second allergen test. The step of determining the treatment endpoint for the allergen based on the results of the first allergen test and the second allergen test may accommodate fractional wheal results.

According to aspects of the disclosure, a method for determining a treatment endpoint for one or more allergens is described. The method includes receiving an input representing a result of a first allergy test; determining, based on the result of the first allergy test, a second allergy test to be administered; receiving an input representing a result of the second allergy test; and determining a treatment endpoint for the one or more allergens based on the result of the second allergy test. Determining the treatment endpoint for the allergen based on the result of the second test accommodates fractional wheal results.

In accordance with some aspects of the disclosure, the first allergy test comprises a multi-prong skin test. The second allergy test comprises an intradermal dilution skin test.

Determining a second allergy test to be administered may include classifying the result of the first allergy test into a plurality of ranges of skin reaction factors, at least one of the plurality of ranges being associated with the second allergy test. The plurality of ranges may include a first range comprising values less than a first endpoint, a second range comprising values greater than or equal to the first endpoint but less than a second endpoint, and a third range comprising values greater than or equal to the second endpoint.

In accordance with some aspects of the disclosure, the result of the first allergy test represents a diameter of a wheal formed in response to the first allergy test.

According to some aspects of the disclosure, the result of the first allergy test may be greater than a defined value, and the treatment endpoint may be determined based on the result of the first allergy test without performing the second allergy test.

In accordance with some aspects of the disclosure, determining a treatment endpoint for the one or more allergens based on the result of the second allergy test may include determining which of a plurality of categories the input representing the result of the second allergy test falls into and selecting the treatment endpoint associated with the determined category. The plurality of categories may each define a range of skin reaction factors.

According to some aspects of the disclosure, a computer-implemented method for determining a treatment endpoint for one or more allergens is described. The method may include receiving, by a processor, a selection of a testing device used to perform a first allergy test; providing a first allergy test result entry mechanism for receiving a result of the first allergy test; receiving, by the processor, the result of the first allergy test; determining, by the processor and based on the result of the first allergy test, at least one parameter for performing a second allergy test; providing a second allergy test result entry mechanism for receiving a result of the second allergy test; and determining, by the processor, a treatment endpoint for at least one allergen based on the result of the second allergy test.

The first allergy test result entry mechanism may be based on a testing protocol specific to the selected testing device.

The first allergy test result entry mechanism may represent a general testing protocol for one or more common allergens.

According to some aspects of the disclosure, the first allergy test comprises a multi-prong skin test. The second allergy test may comprise an intradermal dilution skin test.

In accordance with some aspects of the disclosure, determining at least one parameter for performing the second allergy test may include transmitting the result of the first allergy test to a remote device; and receiving, from the remote device, instructions indicating a need for at least one second allergy test and the at least one parameter for performing the at least one second allergy test.

According to some aspects of the disclosure, the at least one parameter for performing the second allergy test may comprise an intradermal dilution to be used for performing the second allergy test.

In accordance with aspects of the disclosure, the treatment endpoint may be determined based on the result of the first allergy test and the second allergy test. The treatment endpoint for the allergen based on the result of the second test may accommodate fractional wheal results.

According to some aspects of the disclosure, the result of the first allergy test may represent a diameter of a wheal formed in response to the first allergy test. In accordance with some aspects of the disclosure, the first allergy test result entry mechanism may be configured to receive data associated with at least one of a width, height, diameter, color, texture, or sensitivity associated of a wheal forming in response to the first allergy test.

Additional features, advantages, and embodiments of the invention may be set forth or apparent from consideration of the following attached detailed description and drawings. Moreover, it is to be understood that both the foregoing summary of the invention and the following attached detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced.

FIG. 1 shows an example of a method of determining allergen treatment endpoints, in accordance with some aspects of the invention.

FIG. 2 shows a table that includes an example of criteria for selecting allergen treatment endpoints, in accordance with some aspects of the invention.

FIG. 3 depicts an example of a computing device for determining allergen treatment endpoints, in accordance with some aspects of the invention.

FIGS. 4A-4E show examples of screenshots produced on or by a user interface for determining allergen treatment endpoints, in accordance with some aspects of the invention.

### DETAILED DESCRIPTION OF THE INVENTION

The embodiments of the invention and the various features and advantageous details thereof are explained more fully with reference to the non-limiting embodiments and examples that are described and/or illustrated in the accompanying drawings and detailed in the following attached description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention, The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

A system, a method, and an apparatus are described herein for determining skin test sensitivities. In some aspects, a first skin test to evaluate a patient's skin reaction to one or more allergens may be performed. For example, a device, such as a multi-prong skin test device (not shown), may be used to administer the first skin test, as is known by those skilled in the art. After measurements are taken of any reactions to the first skin test, a second skin test may be performed based on the results of the first skin test. In some aspects, the second skin test may include one or more intradermal tests. Based on the results of the second test, an appropriate treatment endpoint for the patient may be determined. In some aspects, a computer-implemented method may be used for determining skin test sensitivities that may guide a user through one or more appropriate test selection and treatment endpoint determinations.

FIG. 1 shows an example of a flowchart of a method for determining skin test sensitivities is shown. As shown at 102, the method may begin when the results of a first skin test to estimate an approximate range of skin reactivity R_{SR} to individual allergens are received. The first skin test may be performed, for example, using a multi-prong skin testing device (not shown). In some aspects, the first skin test results may represent a measurement of the degree of a skin reaction to the applied allergens—i.e., the skin reaction factor (e.g., size, width, height, diameter, color, texture, sensitivity, etc.).

As shown at 104, the results may be classified into a plurality of ranges of skin reaction factors that may be used in determining which second skin test to apply. When using a multi-prong skin testing device to test the reaction to multiple allergens, results for each allergen may be appropriately classified. In some aspects, results may be divided into a first range R₁ comprising values less than a first endpoint EP₁, a second range R₂ comprising values greater than or equal to the first endpoint EP₁ but less than a second endpoint EP₂, and a third range R₃ comprising values greater than or equal to the second endpoint EP,. Of course, the results of the first skin test may be grouped into more or fewer than three ranges of values.

As shown at 106, based on the classification of the results of the first skin test, a second skin test may be selected for one or more of the allergens tested via the first skin test. For example, the second skin test may be an intradermal dilution skin test, and a particular concentration may be selected based on the range in which the first skin test results were classified. In some aspects, the second skin test may be omitted if the first skin test for an allergen falls within a particular range. For example, if the first skin test falls within the highest range of results, this may indicate a positive allergy test result without requiring further testing. Thus, by initially performing the first skin test, the number of intradermal dilution skin tests needed may be reduced.

Based on the results of the second skin test, a treatment endpoint for each allergen may he selected for the patient, as shown at 108. In some aspects, the results of the second skin test may be divided into a plurality of ranges that account for fractional wheals.

FIG. 2 shows a table 200 depicting an example of the how the results of the first and second test may be used to select a treatment endpoint. The values provided in table 200 are merely non-limiting examples of possible categorizations and values. Other categorizations and values are also applicable. The measurements shown in table 200 represent measurements of wheal diameter. In some aspects, other measurements representing a reaction to an allergen may also be used, such as, for example, size, width, height, color, texture, sensitivity, etc.

As shown at 202, the results of the first skin test may be divided into a plurality of ranges (e.g., R₁ to R₃) of skin reaction factors. In the example shown in FIG. 2, the skin reaction factors include ranges of diameters of wheals that appear in response to testing. For instance, the first skin test results may be divided into a first range R₁ comprising measurements less than 3 mm, a second range R₂ comprising measurements greater than or equal to 3 mm and less than 9 mm, and a third range R₃ comprising measurements greater than or equal to 9 mm.

As shown at 204, the table may provide an intradermal dilution ID to be applied in a second test, based on results of the first test. For example, if the result of the first skin test is a wheal of less than 3 mm in diameter (e.g., R₁) intradermal dilution #2 (or ID₂) may be used in a second, intradermal skin test. FIG. 4C shows an example where the intradermal dilution #2 may include, e.g., a dilution for the allergen of White Ash. If the result of the first skin test results in a wheal having a diameter between 3 mm and 9 mm (e.g., R₂), an intradermal dilution #5 (or ID₅) may be applied in the second, intradermal skin test. As seen in FIG. 4C, an example of the intradermal dilution #5 may include a dilution of the allergen for Cats. For those results of the first skin test that include wheals having diameters greater than or equal to 9 mm (e.g., R₃), a positive test may be detected without applying a second skin test.

As shown at 206, the results of the second skin test, such as the intradermal skin test, may be divided into a plurality of categories of ranges of skin reaction factors that may be used to determine a treatment endpoint (shown at 208). As shown at 206, multiple ranges of skin reaction factors for the second skin test may be applied for each intradermal dilution used in the second skin test. For example, where intradermal dilution #2 (or ID₂) was used, intradermal skin reaction factors may be divided into two categories, e.g.: measurements of wheal diameters less than or equal to 6 mm; and measurements of wheal diameters greater than 6 mm. If the resultant skin reaction factors (e.g., wheals) of the second test have diameters less than or equal to 6 mm, the testing may be deemed negative, and no treatment may be necessary. If the resultant skin reaction factors (e.g., wheals) of the second test have diameters greater than 6 mm, the patient may be treated using, e.g., endpoint #3 (or EP₃).

When intradermal dilution #5 (or ID₅) is used to perform the second test, results of the second test that have wheal diameters less than or equal to 6 mm may result in a treatment endpoint #4 (or EP₄); results of the second test that have wheal diameters greater than 6 mm but less than 9 mm may result in a treatment endpoint #5 (or EP₅); and results of the second test that have wheal diameters greater than or equal to 9 mm may result in a treatment endpoint #6 (or EP₆). Those results of the first test that have wheal diameters greater than or equal to 9 mm may also result in the treatment endpoint #6. The ranges of reaction factors shown in table 200 take into consideration fractional measurements of diameters of skin test wheals that may fall between whole number endpoints.

In accordance with some aspects, an apparatus may be provided for determining treatment endpoints for allergy testing. FIG. 3 depicts an example of a computing device 300 that may be used to determine treatment endpoints. The computing device 300 may include a computer. The computing device 300 may include a processor 302 for carrying out processing functions associated with one or more of components and functions described herein. Processor 302 can include a single or multiple set of processors or multi-core processors. Moreover, processor 302 can be implemented as an integrated processing system and/or a distributed processing system.

Computing device 300 further includes a memory 304, such as for storing data used herein and/or local versions of applications being executed by processor 302. For example, memory 304 may be configured to store skin test results. Memory 304 can include any type of memory usable by a computer, such as random access memory (RAM), read only memory (ROM), tapes, magnetic discs, optical discs, volatile memory, non-volatile memory, and any combination thereof Applications may include, for example, one or more object matching applications.

Further, computing device 300 may include a communications component 306 that provides a means for establishing and maintaining communications over one or more communication links with one or more parties utilizing hardware, software, and/or services as described herein. Communications component 306 may carry communications between components on computing device 300, as well as between computing device and external devices, such as devices located across a network and/or devices serially or locally connected to computing device 300. For example, communications component 306 may include one or more buses, and may further include transmit chain components and receive chain components associated with a transmitter and receiver, respectively, operable for interfacing with external devices.

Additionally, computing device 300 may further include a data store 308, which can be any suitable combination of hardware and/or software that provides for mass storage of information and programs employed in connection with aspects described herein. The data store 308 may include a database. For example, data store 308 may be a data repository for applications not currently being executed by processor 302. In some aspects, data store 308 may be located within memory 304.

Computing device 300 may additionally include a user interface component 310 operable to receive inputs from a user of computing device 310, or serving as an application programming interface (API), and may be further operable to generate outputs for presentation to the user. User interface component 310 may include one or more input devices, including but not limited to a keyboard, a number pad, a mouse, a touch-sensitive display, a navigation key, a function key, a microphone, a voice recognition component, a still camera, a video camera, an audio recorder, and/or any other mechanism capable of receiving an input, or any combination thereof. Further, user interface component 310 may include one or more output devices, including but not limited to a display, a speaker, a haptic feedback mechanism, a printer, any other mechanism capable of presenting an output, or any combination thereof. For example, user interface component 310 may be configured to present a series of screen displays for guiding a user through an allergy testing protocol.

Computing device 300 may also include an allergy treatment endpoint determining component 312 configured to determine an appropriate treatment as a result of one or more allergy skin tests. Treatment determining component 312 may be configured to receive inputs representing initial test results, recommend any secondary tests, and recommend treatment endpoints based on the results of any testing performed. In some aspects, allergy treatment endpoint determination component 312 may be configured to implement the method described above with respect to FIG. 1.

The computing device 300 may include a computer-readable medium that includes a plurality of code sections or code segments that, when carried out by the processor 302, cause the computing device 300 to carry out each of the steps described herein. The plurality of code sections or code segments may include a plurality of code section or code segments that are dedicated to specific steps described herein. The computer-readable medium may be provided in the memory 304, or provided as separate software/hardware.

The computing device 300 may be communicatively coupled with a centralized (or decentralized) server and/or database via one or more communication links. The server (not shown) may include a computer-readable medium that includes the plurality of code sections or code segments that, when carried out by the server, cause the server and/or computing device 300 to carry out each of the steps described herein.

FIGS. 4A-4E illustrate a series of example user interface displays that may be presented to a user on, e.g., the computing device 300 when executing a computer implemented method of determining treatments for allergic patients. As shown in FIG. 4A, a user may first be presented with a screen for selecting a testing device for performing an initial skin test. In some aspects, the testing protocol may be device specific. In other aspects, a general testing protocol may be applied irrespective of the testing device in use. The user may select from a plurality of testing devices using, e.g., a drop down menu 402. In some aspects, an “other” option may be presented for selection when the testing device being used is not included in the list.

As shown in FIG. 4B, after selecting a testing device, a screen may be presented that asks the user to enter the results of the initial skin tests. For example, the initial skin test may be performed using a multi-prong skin testing device to apply multiple allergens to a patient's skin. The user may then enter results for each of the tested allergens, as shown in FIG. 4B. In some aspects, an option may be presented fix entering test results for allergens not listed. In this regard, the computing device 300 may be configured to communicate with the server and obtain data and instructions relating to the user-entered allergens. Moreover, the allergens shown in FIG. 4B are merely examples of the types of allergens that may be tested. The systems, methods, and apparatus described herein may also or alternatively be applied to any other allergens.

Upon receiving the results of the initial skin tests, the system (e.g., the computing device 300 and/or server) may determine whether additional testing is needed for any of the allergens tested using the initial skin tests, and determine an intradermal dilution that should be used to perform any second, intradermal skin tests.

As shown in FIG. 4C, the user may be presented with a dialog box that indicates the intradermal dilution that should be used for each allergen that requires a second test.

FIG. 4D depicts an example user interface for entering the results of the intradermal test(s). After entering the results of the intradermal test(s), the user may be presented with a screen, as shown in FIG. 4E, for example, that provides the user with the suggested treatment endpoints based on the results of all tests.

While the invention has been described with regard to an example where the skin reaction factor includes a height of a wheal, the computing device 300 may include fields that are configured to receive data associated with width, diameter, color, texture, sensitivity, etc. of wheals that may result from skin testing.

A “computer,” as used in this disclosure, means any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a super computer, a personal computer, a laptop computer, a palmtop computer, a smart phone, a cellular telephone, a tablet, a web-book, a notebook computer, a desktop computer, a workstation computer, a server, a cloud, or the like, or an array of processors, microprocessors, central processing units, general purpose computers, super computers, personal computers, laptop computers, palmtop computers, notebook computers, desktop computers, workstation computers, servers, or the like.

A “database,” as used in this disclosure, means any combination of software and/or hardware, including at least one application and/or at least one computer. The database may include a structured collection of records or data organized according to a database model, such as, for example, but not limited to at least one of a relational model, a hierarchical model, a network model or the like, The database may include a database management system application (DBMS) as is known in the art. The at least one application may include, but is not limited to, for example, an application program that can accept connections to service requests from clients by sending back responses to the clients. The database may be configured to run the at least one application, often under heavy workloads, unattended, for extended periods of time with minimal human direction

A “network,” as used in this disclosure, means any combination of software and/or hardware, including any machine, device, circuit, component or module, or any system of machines, devices, circuits, components, modules, or the like, which are capable of transporting signals from one location to another location, where the signals may comprise information, instructions, data, and the like. A network may include, but is not limited to, for example, at least one of a local area network (LAN), a wide area network (WAN), a metropolitan area network (MAN), a personal area network (PAN), a campus area network, a corporate area network, a global area network (CAN), a broadband area network (BAN), or the like, any of which may be configured to communicate data via a wireless and/or a wired communication medium.

A “server,” as used in this disclosure, means any combination of software and/or hardware, including at least one application and/or at least one computer to perform services for connected clients as part of aclient-sewer: architecture. The at least one server application may include, but is not limited to, for example, an application program that can accept connections to service requests from clients by sending back responses to the clients. The server may be configured to run the at least one application, often under heavy workloads, unattended, for extended periods of time with minimal human direction. The server may include a plurality of computers configured, with the at least one application being divided among the computers depending upon the workload. For example, under light loading, the at least one application can run on a single computer. However, under heavy loading, multiple computers may be required to run the at least one application. The server, or any of its computers, may also be used as a workstation.

A “communication link,” as used in this disclosure, means a wired and/or wireless medium that conveys data or information between at least two points. The wired or wireless medium may include, for example, a metallic conductor link, a radio frequency (RF) communication link, an Infrared (IR) communication link, an optical communication link, or the like, without limitation. The RF communication link may include, for example, WiFi, WiMAX, IEEE 802.11, DECT, 0G, 1G, 2G, 3G or 4G cellular standards, Bluetooth, and the like. One or more communication links may be used in an environment 100 (shown in FIG. 1) to allow sufficient data throughput and interaction between end-users (such as, e.g., agents, consumers, insurance carriers, estate planners, financial providers, web host providers, and the like). Techniques for implementing such communications links are known to those of ordinary skilled in the art.

The terms “including,” “comprising,” “having,” and variations thereof, as used in this disclosure, mean “including, but not limited to,” unless expressly specified otherwise.

The terms “a,” “an,” and “the,” as used in this disclosure, means “one or more”, unless expressly specified otherwise.

Devices that are in communication with each other need not he in continuous communication with each other, unless expressly specified otherwise. In addition, devices that are in communication with each other may communicate directly or indirectly through one or more intermediaries.

Although process steps, method steps, algorithms, or the like, may be described in a sequential order, such processes, methods and algorithms may be configured to work in alternate orders. In other words, any sequence or order of steps that may be described does not necessarily indicate a requirement that the steps he performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

A “computer-readable medium,” as used in this disclosure, means any medium that participates in providing data (for example, instructions) which may be read by a computer. Such a medium may take many forms, including nonvolatile media, volatile media, and transmission media. Nonvolatile media may include, for example, optical or magnetic disks and other persistent memory. Volatile media may include dynamic random access memory (DRAM). Transmission media may include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer-readable media may be involved in carrying sequences of instructions to a computer. For example, sequences of instruction (i) may be delivered from a RAM to a processor, (ii) may be carried over a wireless transmission medium, and/or (iii) may be formatted according to numerous formats, standards or protocols, including, for example, WiFi, WiMAX, IEEE 802.11, DECT, 0G, 1G, 2G, 3G or 4G cellular standards, Bluetooth, or the like.

While the invention has been described in terms of exemplary embodiments, those skilled in the art will recognize that the invention can be practiced with modifications in the spirit and scope of the appended claims. These examples given above are merely illustrative and are not meant to be an exhaustive list of all possible designs, embodiments, applications or modifications of the invention.

What is claimed:

1. A method for determining a treatment endpoint for one or more allergens, comprising:
receiving an input representing a result of a first allergy test;
determining, based on the result of the first allergy test, a second allergy test to be administered;
receiving an input representing a result of the second allergy test; aid determining a treatment endpoint for the one or more allergens based on the result of the second allergy test,
wherein determining the treatment endpoint for the allergen based on the result of the second test accommodates fractional wheal results.

2. The method of claim 1, wherein the first allergy test comprises a multi-prong skin test.

3. The method of claim 1, wherein the second allergy test comprises an intradermal dilution skin test.

4. The method of claim 1, wherein determining a second allergy test to be administered comprises:
classifying the result of the first allergy test into a plurality of ranges of skin reaction factors, at least one of the plurality of ranges being associated with the second allergy test.

5. The method of claim 4, wherein the plurality of ranges includes a first range comprising values less than a first endpoint, a second range comprising values greater than or equal to the first endpoint but less than a second endpoint, and a third range comprising values greater than or equal to the second endpoint.

6. The method of claim 1, wherein the result of the first allergy test represents a diameter of a wheal formed in response to the first allergy test.

7. The method of claim 1, wherein the result of the first allergy test is greater than a defined value, and wherein the treatment endpoint is determined based on the result of the first allergy test without performing the second allergy test.

8. The method of claim 1, wherein determining a treatment endpoint for the one or more allergens based on the result of the second allergy test comprises:
determining which of a plurality of categories the input representing the result of the second allergy test falls into; and
selecting the treatment endpoint associated with the determined category.

9. The method of claim 8, wherein the plurality of categories each define a range of skin reaction factors.

10. A computer-implemented method for determining a treatment endpoint for one or more allergens, comprising:
receiving, by a processor, a selection of a testing device used to perform a first allergy test;
providing a first allergy test result entry mechanism for receiving a result of the first allergy test;
receiving, by the processor, the result of the first allergy test;
determining, by the processor and based on the result of the first allergy test, at least one parameter for performing a second allergy test;
providing a second allergy test result entry mechanism for receiving a result of the second allergy test; and
determining, by the processor, a treatment endpoint for at least one allergen based on the result of the second allergy test.

11. The method of claim 11, wherein the first allergy test result entry mechanism is based on a testing protocol specific to the selected testing device.

12. The method of claim 11, wherein the first allergy test result entry mechanism represents a general testing protocol for one or more common allergens.

13. The method of claim 11, wherein the first allergy test comprises a multi-prong skin test.

14. The method of claim 11, wherein the second allergy test comprises an intradermal dilution skin test.

15. The method of claim 11, wherein determining at least one parameter for performing the second allergy test comprises:
transmitting the result of the first allergy test to a remote device; and
receiving, from the remote device, instructions indicating a need for at least one second allergy test and the at least one parameter for performing the at least one second allergy test.

16. The method of claim 11, the at least one parameter for performing the second allergy test comprises an intradermal dilution to be used for performing the second allergy test.

17. The method of claim 11, wherein the treatment endpoint is determined based on the result of the first allergy test and the second allergy test.

18. The method of claim 11, wherein determining the treatment endpoint for the allergen based on the result of the second test accommodates fractional wheal results.

19. The method of claim 11, wherein the result of the first allergy test represents a diameter of a wheal formed in response to the first allergy test.

20. The method of claim 11, wherein first allergy test result entry mechanism is configured to receive data associated with at least one of a width, height, diameter, color, texture, or sensitivity associated of a wheal forming in response to the first allergy test.
